(19) United States
(12) Patent Application Publication
(10) Pub. No.: US 2015/0241414 A1
(43) Pub. Date: Aug. 27, 2015

(54) ALKALINE PHOSPHATASE STABILIZER, IMMUNOTEST KIT, PRESERVATION METHOD AND IMMUNOTEST METHOD

(71) Applicant: CANON KABUSHIKI KAISHA, Tokyo (JP)

(72) Inventor: Masaaki Kobayashi, Kawasaki-shi (JP)

(21) Appl. No.: 14/624,838

(22) Filed: Feb. 18, 2015

(30) Foreign Application Priority Data
Feb. 26, 2014 (JP) 2014-035565

(51) Int. Cl. G01N 33/53

(52) U.S. Cl. CPC G01N 33/5306

(57) ABSTRACT

The present invention provides an enzyme stabilizer for stably preserving alkaline phosphatase or an alkaline phosphatase-labeled complex, and an immunotest kit including the stabilizer. The stabilizer is a stabilizer for alkaline phosphatase or an alkaline phosphatase-labeled complex, containing a protein decomposition product as an active ingredient, wherein the decomposition product of the protein has a content of lysine of 50 mg/g or more in the amino acid composition of the product. Further, an embodiment of the present invention provides a method for preserving ALP, using a protein decomposition product which has a lysine content of 50 mg/g or more in the amino acid composition and which contains 95% by weight or more of a protein decomposition having a weight average molecular weight of 10 kDa or less, as a stabilizer.

### BACKGROUND OF THE INVENTION

1. Field of the Invention

The present invention relates to a stabilizer for alkaline phosphatase and an alkaline phosphatase-labeled complex, an immunotest kit including the same, a method for preserving alkaline phosphatase and an immunotest method.

2. Description of the Related Art

Alkaline phosphatase (hereinafter sometimes abbreviated as “ALP”) is an enzyme widely used in an enzyme-linked immunoassay, a chemiluminescent enzyme-linked immunoassay and the like in laboratory tests or as a research reagent, and it is very important in warranting the reagent or the product to stably preserve the enzyme.

It has been known for a long time to add bovine serum albumin (hereinafter referred to as “BSA”) to ALP solutions as a stabilizer for enzymes including ALP; however, it is difficult for the addition of only BSA to lead to the long-term stable preservation of ALP. Thus, various ideas for enzyme stabilization have heretofore been attempted.

Japanese Patent Application Laid-Open No. 2001-183374 describes an improvement in enzyme stability by adding a hydrolysate of gelatin in addition to 1% (w/v) BSA. In addition, Japanese Patent Application Laid-Open No. 2007-151546 describes the addition of a decomposition product of a protein constituting the thread of silk in place of the addition of BSA. Further, Japanese Patent Application Laid-Open No. 2006-42757 describes the addition of a decomposition product of a plant-derived protein in place of the addition of BSA.

A protein is composed of amino acids, and various proteins have their own unique amino acid sequences. The above-described protein decomposition products derived from gelatin, silk and plant primarily contain polypeptides, and these polypeptides probably improve the stability of ALP.

Proteins, however, have their amino acid compositions greatly varying depending on individuals even when the proteins have the same animal or plant origin (species).

Japanese Patent Application Laid-Open No. 2006-42757 describes an enzyme stability test using 3 types of soybean-derived proteins, whose effects have differences in relative merits. In addition, according to the results of research by the present inventors, decomposition products of all of the soybean-derived proteins cannot be said to be effective on the stability of ALP.

An object of the present invention is to provide a stabilizer for more stably preserving ALP in an aqueous solution, an immunotest kit including the stabilizer and a method for preserving ALP.

### SUMMARY OF THE INVENTION

As a result of intensive studies for solving the above problems, the present inventors have found that use of a protein decomposition product having a high content of lysine in the amino acid composition of the product as a stabilizer enables the long-term preservation of ALP or an ALP-labeled complex.

Thus, the present invention is as follows.

(1) A stabilizer for alkaline phosphatase or an alkaline phosphatase-labeled complex, containing a decomposition product of a protein as an active ingredient, wherein the protein decomposition product has a content of lysine of 50 mg/g or more in the amino acid composition.

(2) The stabilizer according to (1), wherein the decomposition product of the protein contains 95% by weight or more of a protein decomposition product having a weight average molecular weight of 10 kDa or less.

(3) The stabilizer according to (1) and (2), wherein the protein decomposition product is derived from soybean, pea or potato.

(4) The stabilizer according to (1) to (3), wherein the protein decomposition product is derived from soybean.

(5) The stabilizer according to (4), wherein the protein decomposition product contains 20% by weight or more of a protein decomposition product having a weight average molecular weight of 1 to 2 kDa.

(6) An immunotest kit, including at least an alkaline phosphatase-labeled complex and the stabilizer according to (1) to (5).

(7) A method for preserving alkaline phosphatase or an alkaline phosphatase-labeled complex, the method including preserving alkaline phosphatase or the alkaline phosphatase-labeled complex in a stabilization solution including the stabilizer according to (1) to (5).

(8) An immunotest method wherein an immunotest is performed using the immunotest kit according to (6).

The present invention enables prevention of inactivation or reduction of ALP enzyme activity and long-term preservation of ALP in an environment of cold storage by using a protein decomposition product which has a content of lysine of 50 mg/g or more in the amino acid composition and which contains 95% by weight or more of a protein decomposition product having a weight average molecular weight of 10 kDa or less, as a stabilizer. The present invention also enables, in the selection of a stabilizer for ALP, the change of its lot, or the like, the easy determination of whether the stabilizer is a material suitable for ALP preservation by examining the content of lysine and the molecular weight distribution of a protein decomposition product to be used. Thus, a material suitable for ALP preservation can be selected from among various animal- and plant-derived proteins for each application.

An embodiment of the present invention is characterized in that the protein decomposition product is derived from soybean, pea or potato; for these plants, the use of each plant-derived protein decomposition product as a stabilizer enables the provision of a stabilizer which is safe and causes no concern about a zoonotic infection (bovine spongiform encephalopathy: BSE) and the like.

Another embodiment of the present invention provides an immunotest kit including the stabilizer of the present invention.

Further features of the present invention will become apparent from the following description of exemplary embodiments with reference to the attached drawings.

### BRIEF DESCRIPTION OF THE DRAWINGS

FIG. 1 is a conceptual diagram of an analyzer used in Examples 1 and 2 of the present invention.

FIG. 2 is a conceptual diagram of an insoluble support used in Examples 1 and 2 of the present invention.

FIG. 3 is a conceptual diagram of an immunotest kit used in Examples 1 and 2 of the present invention.

FIG. 4 is a graph showing the results of studies performed in Example 2 of the present invention.

### DESCRIPTION OF THE EMBODIMENTS

Preferred embodiments of the present invention will now be described in detail in accordance with the accompanying drawings.

The embodiments of the stabilizer for alkaline phosphatase and an alkaline phosphatase-labeled complex and the immunotest kit including the stabilizer according to the present invention will be described below. The embodiments shown herein are only illustrative and the present invention is not necessarily intended to be limited to these embodiments.

### First Embodiment

A first embodiment of the present invention provides a stabilizer for alkaline phosphatase or an alkaline phosphatase-labeled complex, containing a decomposition product of a protein as an active ingredient, wherein the decomposition product of the protein has a content of lysine of 50 mg/g or more in the amino acid composition.

Alkaline phosphatase (ALP) is an enzyme which has an optimal pH in the alkaline region and can hydrolyze many phosphoester bonds, and is broadly present in various organism species. As described above, ALP is widely used in an enzyme-linked immunoassay, a chemiluminescent enzyme immunoassay, and the like in laboratory tests or as a research reagent.

Lysine refers to 2,6-diamino-n-caproic acid, and is a basic amino acid abbreviated as Lys or K.

In this embodiment, the phrase “the decomposition product of the protein has a content of lysine of 50 mg/g or more in the amino acid composition” means that free or non-free lysine is contained in an amount equivalent to 50 mg or more per 1 g of the protein decomposition product. More specifically, the content of lysine is based on amino-acid composition analysis, and examples of the amino-acid composition analysis include a ninhydrin method. The stabilizer of this embodiment meets the requirement that the decomposition product of a protein as an active ingredient has a content of lysine of 50 mg/g of the product or more in the amino acid composition. Examples of the “protein” for the stabilizer of the present invention include various proteins extracted from cereals, tubers and roots, pulses, nuts and seeds, vegetables, fruits, mushrooms, algae, fish and seafood, meats, eggs, and milks. Among decomposition products of these proteins, products having a content of lysine of 50 mg/g or more in the amino acid composition can each be used as the stabilizer of the present invention. In addition, a higher content of lysine in such a protein decomposition product is better, and 55 mg/g or more is more preferable. The content of lysine in a protein decomposition product is not always proportional to the lysine content of such a food itself. For example, although lysine is known to be poorly contained in fruit species, such as pear, apple and orange, and cereals, such as rice, corn and wheat, some proteins derived therefrom meet that their decomposition products each have a content of lysine of 50 mg/g of the product or more per 1 g of the protein decomposition product, and such proteins can naturally be said to be suitable as the stabilizers for ALP according to the present invention. According to the present invention, the origin of the protein is not limited; however, the protein is preferably derived from soybean, pea or potato, and more preferably derived from soybean.

The “decomposition product” of a protein means a mixture of substances obtained by the decomposition of the protein, and contains polypeptides, peptides, amino acids, salts, and the like and mainly contains polypeptides. In this embodiment, the decomposition product of a protein can preferably contain 95% by weight or more of substances having a weight average molecular weight of 10 kDa or less in the molecular weight distribution of the decomposition product, and more preferably, the decomposition product of the protein contains 95% by weight or more of substances having a weight average molecular weight of 10 kDa or less and 20% by weight or more of substances having a weight average molecular weight of 1 to 2 kDa.

The method for obtaining the decomposition product of a protein is not particularly limited, and examples of the method include acid decomposition, enzymolysis and alkali decomposition.

The enzyme usable for the decomposition of a protein may be an enzyme which belongs to protease and can decompose the protein mainly into polypeptides. Examples of the enzyme include animal-derived proteases, such as pepsin, trypsin and chymotrypsin, and plant-derived proteases, such as papain, ficin and bromelin. Examples of the acid for the acid decomposition include hydrochloric acid, sulfuric acid, phosphoric acid and nitric acid, and examples of the alkali for alkali decomposition include sodium hydroxide and potassium hydroxide.

Although polypeptides of various lengths with weight average molecular weights of 10 kDa or less are obtained by the decomposition of a protein, the decomposition product according to the present invention may contain a mixture in which polypeptides of various lengths are mixed, or may contain polypeptides fractionated to the range of a certain length. The decomposition of a protein may simultaneously yield amino acids; however, amino acids and other substances derived from the protein may be mixed in the stabilizer of the present invention provided that the polypeptides are contained in the stabilizer. As used herein, the polypeptide refers to any polypeptide in which a plurality of amino acids are bonded to each other, and the number of amino acid units, the molecular weight and the steric structure do not matter.

According to the present invention, the stabilizer is prepared into a solution and used to prevent the inactivation and activity reduction of ALP or an ALP-labeled complex. The stabilizer-containing solution is called a stabilization solution.

Various aqueous solutions can each be used for the preparation of the stabilization solution. Examples of such aqueous solutions include physiological saline and phosphate buffered physiological saline; however, Good's buffer is particularly preferable. In addition, the pH of the buffer may be used by properly selecting suitable pH and is selected in the range of pH 3 to 9, preferably pH 6 to 8, more preferably pH 6.3 to 6.8. The buffer may properly contain one or more selected from the group consisting of saccharides, polyethylene glycol, metal salts, such as magnesium chloride and zinc chloride, preservatives, such as sodium azide or antibiotics, and surfactants, such as non-ionic surfactants, ampholytic surfactants or anionic surfactants.

The concentration of the protein decomposition product in the stabilization solution according to the present invention may be in the range of 0.01 to 20% (w/v), more preferably 0.1 to 10% (w/v).

The ALP-labeled complex refers to a compound in which ALP bonds to a substance to be labeled. The substance to be labeled is not particularly limited. In view of the general intended use of ALP, examples of the substance to be labeled include a substance capable of specifically recognizing a biological material; specific examples of the substance include one of a pair of substances, such as antigen and antibody, biotin and avidin, or biotin and streptavidin, a specific saccharide and a corresponding lectin, a cytokine or chemokine and a corresponding receptor, and an endotoxin and an endotoxin-neutralizing protein. When the substance to be labeled is an antibody, examples of the antibody include antibodies, polyclonal antibodies and monoclonal antibodies acquired from various immune animals, such as mice, rats, rabbits and goats, or fragments of these antibodies, such as Fab, F(ab′)2 and Fab′.

Although the present invention is a stabilizer for ALP or an ALP-labeled complex, the stabilizer of the present invention is not limited to for ALP and can also be used as a stabilizer for other enzymes or proteins.

### Second Embodiment

A second embodiment of the present invention provides an immunotest kit including at least alkaline phosphatase or an alkaline phosphatase-labeled complex diluted with a stabilization solution containing the above-described stabilizer.

The immunotest refers to all tests utilizing antigen-antibody reaction, and examples of the test include tests based on well-known methods, such as an enzyme-linked immunosorbent assay or an enzyme immunoassay (ELISA or EIA), a chemiluminescent enzyme immunoassay (CLEIA) and a luminescence enzyme immunoassay. For example, the chemiluminescent enzyme immunoassay can be performed using an insoluble support on which a first antibody is solid-phased, and an ALP-labeled second antibody dilutes with the stabilizer, a washing buffer solution, and a luminescent/chromogenic substrate solution. The detection of a signal is performed by reacting the substrate with ALP labeled on the second antibody under optimal conditions and measuring the amount of the enzyme reaction product by an optical method. The immunotest may be performed by a hand method or may be carried out using an apparatus, such as an analyzer.

The insoluble support used for the immunotest may be a solid-phase support having a shape, such as a bead, a microplate, a test tube, a stick or a membrane, made of a material, such as polystyrene, polycarbonate, polyvinyl toluene, polypropylene, polyethylene, polyvinyl chloride, nylon, polymethacrylate, polyacrylamide, latex, liposome, gelatin, agarose, cellulose, sepharose, glass, metal, ceramic, or magnetic substance.

The method for solid-phasing the first antibody on the insoluble support may be a well-known solid-phasing method. For example, physical adsorption methods include a method involving mixing and contacting the antibody with the support in a solution, such as a buffer solution, and a method involving contacting the support with the antibody dissolved in a buffer solution or the like.

The solid-phasing of the first antibody by a chemical binding method can be performed according to a well-known method. Examples of the method include a method which involves mixing and contacting the antibody and the support with a divalent cross-linking reagent, such as glutaraldehyde, carbodiimide, imide ester or maleimide for reaction with the amino group, the carboxyl group, the thiol group, the aldehyde group, the hydroxyl group or the like of both of the antibody and the support.

Further, if it is necessary to perform treatment for suppressing non-specific reaction, the spontaneous agglutination of the insoluble support on which the first antibody is solid-phased, or the like, the treatment can be carried out by a well-known method. Examples of the method include a method which involves contacting the surface or inner wall surface of the insoluble support on which the antibody is solid-phased, with a protein, such as bovine serum albumin (BSA), casein, gelatin, ovalbumin or a salt thereof, a surfactant, skim milk, or the like for coating.

The method for labeling ALP on the second antibody may be a well-known labeling method. Examples of the method include a method which involves mixing and contacting the antibody and ALP with a divalent cross-linking reagent, such as glutaraldehyde, carbodiimide, imide ester or maleimide for reaction with the amino group, the carboxyl group, the thiol group, the aldehyde group, the hydroxyl group or the like of both of the antibody and ALP.

The composition of the immunotest kit of the present invention is not particularly limited provided that the ALP-labeled complex solution contains a stabilizer containing a decomposition product of a protein as an active ingredient, and the kit may include various reagents necessary for the immunotest in addition to the ALP-labeled complex solution. Examples of such reagents include a reagent containing a buffer solution, a reagent dilution and a substance generating a signal, such as coloring, a reagent containing a substance involved in the generation of a signal, such as coloring, a reagent containing a substance for calibration, and a reagent containing a substance for accuracy control.

As an example of the form of a disposable immunotest kit, a configuration can be employed in which a test container is packed with a spherical or rodlike insoluble support on which the first antibody is solid-phased, a reagent dilution, the ALP-labeled second antibody diluted with a stabilizer, a wash solution, a luminescent substrate solution and the like.

The shape of the test container is not particularly limited provided that a sample can be measured. Examples of the container include a scaphoid container in which pluralities of reaction vessels and reagent-housing vessels are arranged, and a channel-type container in which grooves are provided on a plate-like base substrate with reaction vessels and housing vessels connected through channels. The size of the test container is also not particularly limited; however, the container can have a small size of on the order of 10 centimeters×10 centimeters or less to use the container by incorporation in an automatic analyzer or the like.

In addition, the top of each vessel may also be sealed to avoid the contamination of the reaction vessel with foreign materials and the evaporation/deterioration of the reagent packed in the reagent-housing vessel. For example, there is a method which involves causing an aluminum foil, a polymer film, or the like to adhere to the top of each of the reaction vessel and the housing vessel of the test container. The sealing with an aluminum foil is particularly preferable because unsealing can be easily performed at the extremity of a punching station or a dispensation tip in the analyzer. The material of the test container is not particularly limited provided that the material is not a substance inhibiting reaction for measuring the substance to be measured. Examples of the material include polystyrene resin, polyethylene resin and polypropylene resin.

The following configuration is possible as the form of the microplate-type test kit. For example, there is a configuration with which a microplate having the first antibody solid-phased thereon as well as a sample dilution, the ALP-labeled second antibody diluted with a stabilizer, a wash solution, a chromogenic substrate solution and the like are included in the form of reagent bottles.

### Third Embodiment

A third embodiment of the present invention provides a method for preserving alkaline phosphatase or an alkaline phosphatase-labeled complex, the method including preserving alkaline phosphatase or the alkaline phosphatase-labeled complex in a stabilization solution containing the above-described stabilizer. The concentration of the protein decomposition product in the stabilization solution according to this embodiment is preferably in the range of 0.01 to 20% (w/v), more preferably 0.1 to 10% (w/v).

### EXAMPLES

The present invention will be specifically described below with reference to Examples. However, the following Examples are only illustrative of the present invention, and the scope of the present invention is not intended to be limited to the following Examples in any manner.

(1) Analyzer for Immunotest Kit

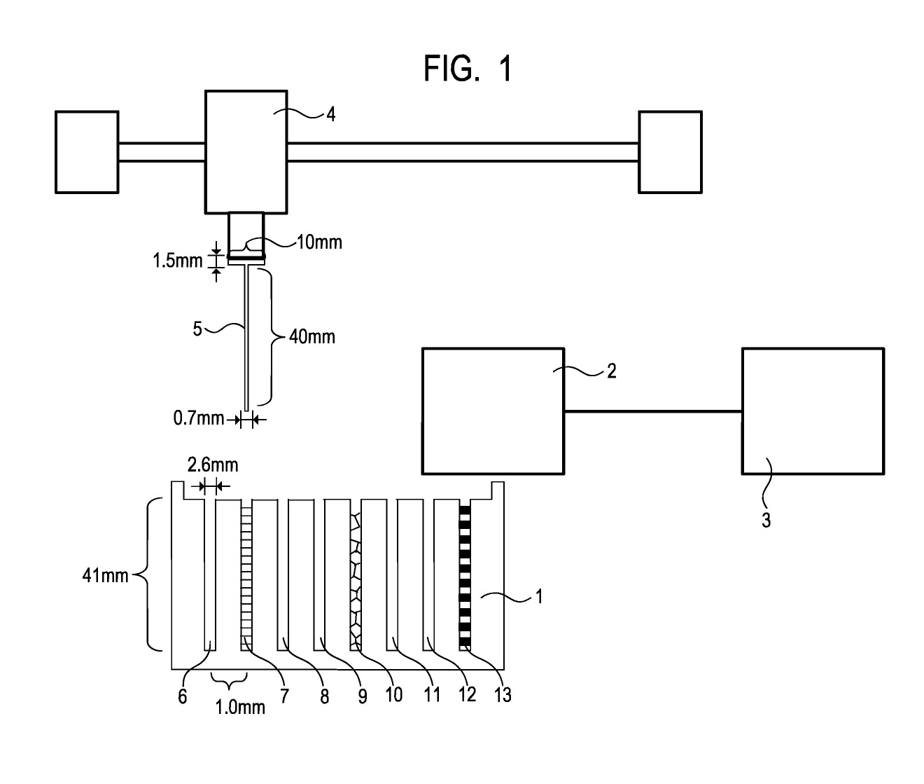

FIG. 1 shows a conceptual diagram of the analyzer for the immunotest kit prepared in this Example. As shown, a test container 1 is incorporated in the analyzer. In the same figure, 2 is a photomultiplier; 3 is a photon counter; and 4 is a handling arm for transferring an insoluble support 5. As shown, the photomultiplier 2 is disposed above the test container 1 to receive light from the luminescent reaction produced in measuring a substance to be measured. The photon counter 3 is placed to measure a signal from the photomultiplier 2 and connected to an external output apparatus. As shown, the handling arm 4 is used in transferring the insoluble support 5 and disposed above the test container 1.

(2) Preparation of Insoluble Support

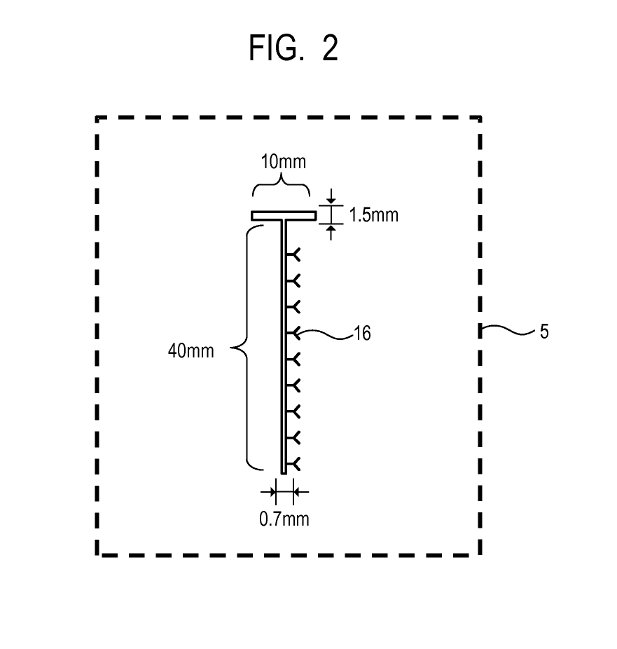

The insoluble support 5 described in FIG. 2 was prepared by injection-molding a polystyrene resin. The tabular region of the upper part of the support is a tabular region used in transfer by the handling arm 4 and made in a diameter of 10 mm and a thickness of 1.5 mm. The columnar region of the insoluble support 5 is a region for solid-phasing an antibody 16 specifically recognizing a substance to be measured thereon and made in a diameter of 0.7 mm and a length of 40 mm.

(3) Preparation of Test Container

The test container 1 described in FIG. 1 was prepared by injection-molding a polypropylene resin. The test container is provided with a plurality of reaction vessels for performing an immunotest. Each vessel was made in a diameter of 2.6 mm and a depth of 41 mm. The interval between the contiguous vessels was 10 mm.

(4) Method for Transferring Insoluble Support

The end of the handling arm 4 was equipped with a tubular jig having a silicone rubber-made cylindrical end and a tube (not shown). In addition, the tip of the tube was equipped with a magnetic valve and an air pump. The insoluble support 5 was made liftable/releasable via the tabular region by making the inner part of the tube in negative pressure by the air pump and turning the magnetic valve on and off. The handling arm 4 was moved by the mechanism to enable the insoluble support 5 to move from vessel to vessel in the test container 1.

(5) Solid-Phasing of First Anti-IL-6 Antibody on Insoluble Support

An anti-IL-6 monoclonal antibody (R&D Systems Co., Ltd.) was diluted to 10 μg/ml with Tris buffer (pH 8.0), and 100 μL was dispensed into each container for solid-phasing the antibody. Thereafter, the columnar region of the insoluble support 5 was immersed in the solution and allowed to stand at 4° C. overnight. Then, the insoluble support 5 was taken out and the surface thereof was washed with Tris buffer (pH 8.0). Next, bovine serum albumin (Sigma-Aldrich Japan Co., Ltd.) was diluted to 5% (by weight) with Tris buffer (pH 8.0), and 100 μL was dispensed into each container for solid-phasing the antibody. Thereafter, the columnar region of the insoluble support 5 was immersed in the solution and allowed to stand at room temperature for 2 hours. Then, the insoluble support 5 was taken out and the surface thereof was washed with Tris buffer (pH 8.0). Then, sucrose (Wako Pure Chemical Industries Ltd.) was diluted to 5% (w/v) with Tris buffer (pH 8.0), and 100 μL was dispensed into each container for solid-phasing the antibody. Subsequently, the columnar region of the insoluble support 5 was immersed in the solution and allowed to stand at room temperature for 2 hours. Finally, the insoluble support 5 was taken out, dried and preserved at 4° C.

(6) Solid-Phasing of First Anti-CRP Antibody on Insoluble Support

An insoluble support 5 on which an anti-CRP monoclonal antibody (Oriental Yeast Co., Ltd.) was solid-phased was prepared by the same procedure in (5) above.

(7) Preparation of Stabilization Solution

A buffer solution was prepared by adding 2-morpholinoethanesulfonic acid, monohydrate (25 mM), sodium chloride (150 mM), magnesium chloride (1 mM), zinc chloride (0.1 mM) and sodium azide (0.09%) to pure water.

Then, 9 plant protein decomposition products (Sigma-Aldrich Japan Co., Ltd.) as shown in Table 1 were each added to 5% (w/v) to the prepared buffer solution, which was then adjusted to pH 6.5 with sodium hydroxide, and the resultant solutions were used as stabilization solutions for ALP (Nos. 1 to 9). In addition, 4 animal protein decomposition products (Nippi Co., ltd.) as shown in Table 1 were each added to 10% (w/v) to the prepared buffer solution, which was then adjusted to pH 6.5 with sodium hydroxide, and the resultant solutions were used as stabilization solutions for ALP (Nos. 10 to 13).

To perform performance comparison with a protein non-decomposition product, a solution was prepared in which 5% (w/v) bovine serum albumin (Sigma-Aldrich Japan Co., Ltd.) was added to the prepared buffer solution (control).

The reagents as shown in Table 1 are protein decomposition products obtained by enzyme and acid decomposition, and each contained 95% by weight or more of polypeptides having weight average molecular weights of 10 kDa or less.

In addition, the soybean protein decomposition product of No. 2 had a molecular weight distribution in terms of weight ratio of 0.5% for 10 kDa or more, 3.1% for 5 to 10 kDa, 13% for 2 to 5 kDa, 20.5% for 1 to 2 kDa, 25.2% for 0.5 to 1 kDa and 37.7% for 0.5 kDa or less.

TABLE 1

| | | Protein Decomposition Product | Decomposition |
|---|---|---|---|
| Number | Origin | (Reagent Name) | Method |
| No. 1 | Corn | Gluten Hydrolysate from maize | Enzyme |
| No. 2 | Soybean | Peptone N-Z-Soy BL7 | Enzyme |
| No. 3 | Pea | Peptone from pea | Enzyme |
| No. 4 | Soybean | Peptone from soybean, enzymatic | Enzyme |
| | | digest | |
| No. 5 | Wheat | Wheat Peptone | Enzyme |
| No. 6 | Broad | Broadbean Peptone | Enzyme |
| | Bean | | |
| No. 7 | Soybean | Soy protein acid hydrolysate | Acid |
| No. 8 | Soybean | Peptone from Glycine max | Enzyme |
| | | (soybean) | |
| No. 9 | Potato | Peptone from potatoes | Enzyme |
| No. 10 | Cow | Bovine Hide Collagen Peptide | Enzyme |
| | | (AFC) | |
| No. 11 | Pig | Pig Hide Collagen Peptide (PS-1) | Enzyme |
| No. 12 | Fish | Fishskin Collagen Peptide (FCP) | Enzyme |
| No. 13 | Fish | Fishskin Collagen Peptide (FCP-A) | Enzyme |
| Control | Albumin | Albumin from bovine serum | Non- |
| | | | decomposition |

(8) Labeling of ALP on Second Anti-IL-6 Antibody

Enzyme labeling on an anti-IL-6 monoclonal antibody (R&D Systems Co., Ltd.) was performed using Alkaline Phosphatase Labeling Kit-NH2 (trade name, Dojindo Laboratories) according to the maker's protocol.

Then, the ALP-labeled anti-IL-6 monoclonal antibody was diluted to 2.0 μg/ml using each of 13 stabilizers or a control solution as shown in Table 1, followed by preservation at 4° C.

(9) Labeling of ALP on Second Anti-CRP Antibody

An ALP-labeled anti-CRP monoclonal antibody was prepared by the same procedure as in (8) above. Then, the ALP-labeled anti-CRP monoclonal antibody was adjusted to 2.0 μg/ml using No. 7 (soybean protein decomposition product) shown in Table 1, followed by preservation at 4° C.

(10) Preparation of Immunotest Kit

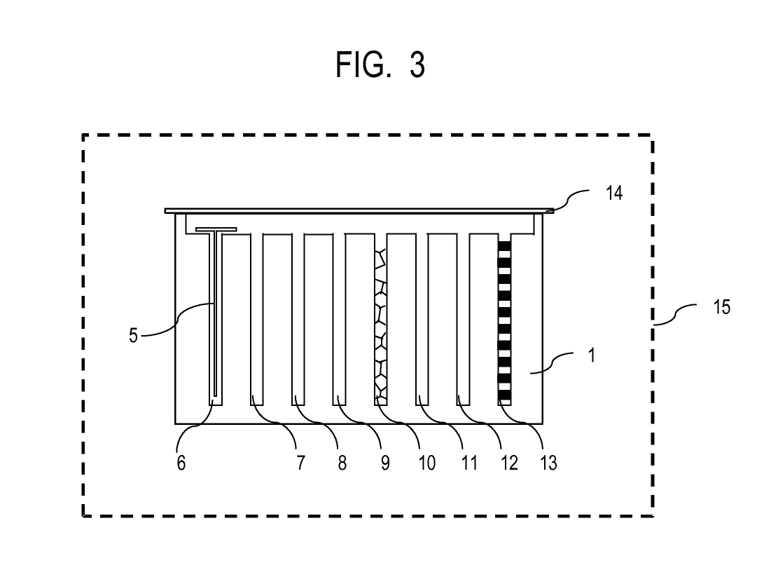

FIG. 3 shows a conceptual diagram of an immunotest kit 5 used in these Examples.

The insoluble support 5, shown in each of (5) and (6), on which the antibody was solid-phased was inserted into a vessel 6 for disposing the insoluble support in the test container 1. Then, 0.05% (w/v) Tween 20-containing Tris buffer (pH 8.0) was used as a wash solution to dispense 115 μL thereof into each of washing vessels 8, 9, 11 and 12. Next, 100 μL each of the 2.0 μg/ml ALP-labeled antibody solutions shown in the operations (8) and (9) were each dispensed into an ALP-labeled antibody reagent vessel 10. Subsequently, 100 μL of a chemiluminescent reagent (Lumigen Co., Ltd.) was dispensed into a luminescent reaction vessel 13. Finally, a laminated film 14 was thermocompressed on the test container 1, and the resultant was used as an immunotest kit 15.

In using the immunotest kit 15, the laminated film 14 was peeled off and 100 μL of a sample was dispensed into an immune reaction vessel 7. Thereafter, the immunotest kit was placed in the analyzer shown in FIG. 1 for measurement.

(11) Amino Acid Composition of Protein Decomposition Product Used in Each Stabilizer

The amino acid composition of the 9 protein decomposition products (Nos. 1 to 9) and albumin (protein non-decomposition product) used in the stabilizers of (7) above was analyzed. The amino acid composition analysis was carried out as follows.

First, about 2 mg each of the above samples (Nos. 1 to 9 and albumin) were each taken into a test tube, to which 250 μL of 6 mol/L hydrochloric acid was then added, followed by sealing the tube under reduced pressure after replacement with nitrogen. Then, hydrolysis was performed at 110° C. for 22 hours, followed by exsiccation under reduced pressure. Next, 400 μL of 0.02 mol/L hydrochloric acid was added to the test tube in which each sample was exsiccated for the dissolution of the sample, followed by filtration with a 0.22-μm centrifugal filtration unit. Subsequently, the filtrate was recovered and diluted 10 times in 0.02 mol/L hydrochloric acid, and 50 μL of the sample solution was transferred to a sample cup for amino acid analysis. Thereafter, each sample solution was placed in an amino acid analyzer L-8900 (Hitachi High-Technologies Corporation) and subjected to quantitative amino acid analysis by the ninhydrin method.

The amino acid composition of the protein decomposition products (Nos. 1 to 9) and albumin (protein non-decomposition product) is shown in Table 2. The values described in the table each represent the content (mg/g) of each amino acid in 1 g of each protein decomposition product (sample).

As shown in Nos. 2, 4, 7 and 8, even the soybean-derived protein decomposition products were greatly different in the content of each amino acid in each protein decomposition product (1 g). For example, the content of lysine was 56 mg/g, 36 mg/g, 50 mg/g and 35 mg/g for Nos. 2, 4, 7 and 8, respectively. The content of lysine in each of the protein decomposition products of Nos. 2, 3, 7 and 9 was 50 mg/g or more. In contrast, the content of lysine in albumin (protein non-decomposition product) as control was as high as 109 mg/g.

TABLE 2

| | | Amino acid Composition (mg/g) | | | | | | | | | | | | | | | |
|---|---|---|---|---|---|---|---|---|---|---|---|---|---|---|---|---|---|
| Number | Origin | Asp | Thr | Ser | Glu | Gly | Ala | Val | Met | Ile | Leu | Tyr | Phe | Lys | His | Arg | Pro |
| No. 1 | Corn | 39 | 20 | 25 | 120 | 18 | 47 | 28 | 12 | 22 | 79 | 23 | 31 | 19 | 9 | 12 | 51 |
| No. 2 | Soybean | 107 | 31 | 42 | 201 | 32 | 32 | 36 | 10 | 37 | 59 | 27 | 38 | 56 | 20 | 68 | 44 |
| No. 3 | Pea | 94 | 28 | 37 | 149 | 29 | 30 | 37 | 7 | 34 | 54 | 28 | 41 | 62 | 20 | 77 | 34 |
| No. 4 | Soybean | 68 | 21 | 26 | 113 | 41 | 23 | 24 | 7 | 23 | 39 | 20 | 25 | 36 | 13 | 43 | 28 |
| No. 5 | Wheat | 20 | 15 | 23 | 260 | 19 | 15 | 25 | 10 | 23 | 45 | 21 | 36 | 10 | 13 | 22 | 86 |
| No. 6 | Broad Bean | 66 | 20 | 23 | 106 | 22 | 22 | 27 | 4 | 25 | 43 | 20 | 27 | 40 | 15 | 59 | 25 |
| No. 7 | Soybean | 75 | 16 | 12 | 153 | 28 | 51 | 38 | 9 | 35 | 58 | 26 | 39 | 50 | 16 | 60 | 41 |
| No. 8 | Soybean | 67 | 22 | 23 | 109 | 21 | 23 | 27 | 7 | 25 | 41 | 21 | 28 | 35 | 14 | 40 | 27 |
| No. 9 | Potato | 90 | 36 | 29 | 74 | 29 | 31 | 41 | 14 | 39 | 65 | 38 | 41 | 53 | 15 | 36 | 35 |
| Control | Albumin | 68 | 51 | 42 | 106 | 16 | 53 | 56 | 9 | 25 | 106 | 47 | 62 | 109 | 31 | 56 | 40 |

### Example 1

In Example 1, a preservation test of an ALP-labeled anti-IL-6 monoclonal antibody solution was performed using the 13 stabilization solutions and the control solution prepared in (7) above.

The above-described analyzer was used for measurement. Immunoassay kits for measuring IL-6 were prepared according to (5), (7), (8) and (10) above and stored at 4° C. As a sample, an IL-6 (Kamakura Techno-Science Inc.) solution adjusted to a concentration of 160 ng/ml was preserved in portions at −30° C. and used after thawing in each measurement.

In a measurement method, the immunotest kits having been stored at 4° C. were first allowed to stand at room temperature for 30 minutes or more. The laminated film was then peeled off from the immunotest kit, and 100 μL of a control or a sample were dispensed at the position of the immune reaction vessel 7. The immunotest was performed using a 2-step sandwich CLEIA method.

The 2-step sandwich CLEIA was carried out at 37° C. The IL-6-solid-phased insoluble support was first immersed in the immune reaction vessel 7 and allowed to stand for 10 minutes. The insoluble support was then immersed in the washing vessel 8 and allowed to stand for 2 minutes, and the support was immersed in the washing vessel 9 and allowed to stand for 2 minutes. The insoluble support was subsequently immersed in the ALP-labeled antibody reagent vessel 10 and allowed to stand for 7.5 minutes. Thereafter, the insoluble support was immersed in the washing vessel 11 and allowed to stand for 2 minutes, and the support was immersed in the washing vessel 12 and allowed to stand for 2 minutes. Then, the insoluble support was immersed in the luminescent reaction vessel 13 and allowed to stand for 3 minutes. Finally, the insoluble support was pulled out of the luminescent reaction vessel 13, and the luminescence intensity of the chemiluminescent reagent was measured.

The preservation test was carried out as follows. The signal value obtained in measuring the sample at the 1st day from the start of immunotest kit preservation was used as a reference value. Thereafter, the measurement of the sample using each immunotest kit was performed at about 10-day intervals, and the value obtained by dividing the resultant signal value by the reference value was defined as a remaining activity rate (%).

The evaluation method involved calculating the remaining activity rate for each cartridge at 30, 60 and 90 days after the start of preservation and evaluating the remaining activity rate of 90% or more at the time of 60 days as “+” and the remaining activity rate of 90% or more at the time of 90 days as “++”.

As a result, the stabilizers having a remaining activity rate of 90% or more at 60 days after the start of preservation were of Nos. 2, 3, 7 and 9, and all of the stabilizers had contents of lysine as a basic amino acid of 50 mg/g or more.

The stabilizers having a remaining activity rate of 90% or more at 90 days after the start of preservation were of Nos. 2 and 3, and had contents of lysine as a basic amino acid of 55 mg/g or more.

In contrast, albumin used as control has a lysine content of 109 mg/g in the composition thereof and is a protein non-decomposition product (weight average molecular weight: 66 kDa), and had remaining activity rates of as low as 90% or less at 60 and 90 days thereafter.

The animal protein decomposition products (collagen peptides) used in Nos. 10 to 13, which are polypeptides containing glycine, proline and hydroxyproline as the main components, has a low lysine content and all had low remaining activity rates at 30 days thereafter.

As described above, the alkaline phosphatase-labeled complex could be stably preserved by using the protein decomposition products having lysine contents of 50 mg/g or more and containing 95% by weight or more of polypeptides having weight average molecular weights of 10 kDa or less in the molecular distribution.

TABLE 3

| Number | Origin | Day 30 | Day 60 | Day 90 | Evaluation |
|---|---|---|---|---|---|
| No. 1 | Corn | 81.9 | 67.5 | 56.9 | − |
| No. 2 | Soybean | 98.7 | 96 | 91.8 | ++ |
| No. 3 | Pea | 97.2 | 94.2 | 91 | ++ |
| No. 4 | Soybean | 87.5 | 79.1 | 74.8 | − |
| No. 5 | Wheat | 93.7 | 88.2 | 83.7 | − |
| No. 6 | Broad Bean | 84.6 | 72.4 | 63.5 | − |
| No. 7 | Soybean | 98.5 | 94.4 | 87.5 | + |
| No. 8 | Soybean | 90.8 | 83.3 | 77.7 | − |
| No. 9 | Potato | 98 | 93.5 | 86.4 | + |
| No. 10 | Cow | 85 | — | — | − |
| No. 11 | Pig | 77.8 | — | — | − |
| No. 12 | Fish | 77.6 | — | — | − |
| No. 13 | Fish | 85.4 | — | — | − |
| Control 1 | Albumin | 92.3 | 84.9 | 77.9 | − |

Numerical Value: Remaining Activity (%)

### Example 2

In Example 2, an immunotest kit for CRP measurement was prepared using the stabilizer of No. 7, and the preservation stability was examined at 4° C.

The above-described analyzer was used for the measurement. Immunoassay kits for CRP measurement was prepared according to (6), (7), (9) and (10) above and stored at 4° C. As a sample, a CRP (Oriental Yeast Co., Ltd.) solution adjusted to a concentration of 0.15 mg/dl was preserved in portions at −30° C. and used after thawing in each measurement.

In a measurement method, the immunotest kits having been stored at 4° C. were each first allowed to stand at room temperature for 30 minutes or more. The laminated film was peeled off from the immunotest kit, and 100 μL of a control sample was dispensed into the immune reaction vessel 7. The immunotest was performed using the 2-step sandwich CLEIA method described in Example 1.

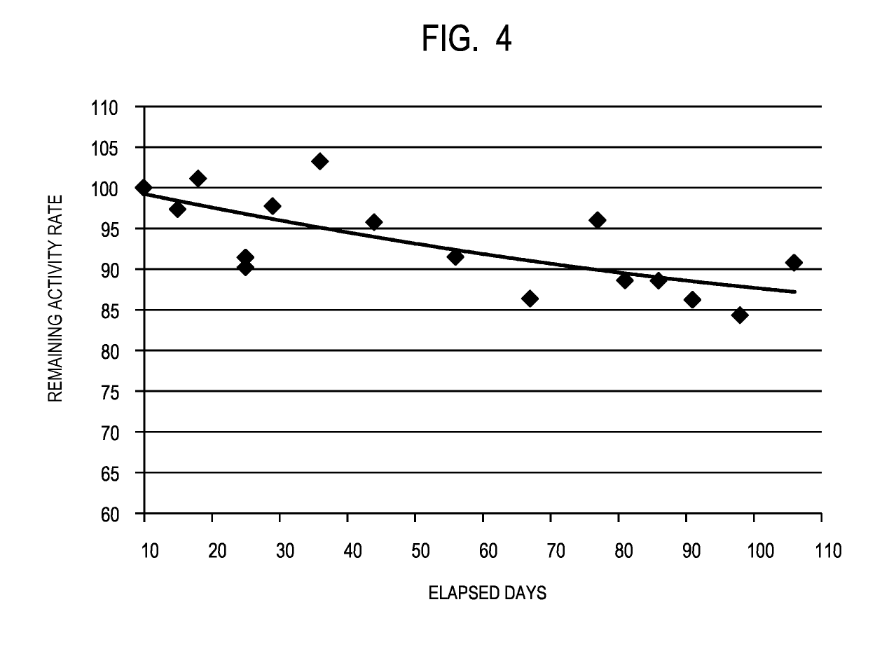

The results are shown in FIG. 4. The remaining activity rate at the 60th day from the start of preservation was 91.9%, and the remaining activity rate at the 90th day was 88.7%. These results support the results of Table 3 (No. 7), and showed that the decomposition product of the protein having a lysine content of 50 mg/g or more in the amino acid composition could be used to stably preserve the alkaline phosphatase-labeled complex.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-035565, filed Feb. 26, 2014, which is hereby incorporated by reference herein in its entirety.

What is claimed is:

1. A stabilizer for alkaline phosphatase or an alkaline phosphatase-labeled complex, comprising a decomposition product of a protein as an active ingredient, wherein the decomposition product of the protein has a content of lysine of 50 mg/g or more in the amino acid composition.

2. The stabilizer according to claim 1, wherein the decomposition product of the protein comprises 95% by weight or more of a protein decomposition product having a weight average molecular weight of 10 kDa or less.

3. The stabilizer according to claim 2, wherein the protein is derived from soybean, pea or potato.

4. The stabilizer according to claim 2, wherein the protein is derived from soybean.

5. The stabilizer according to claim 4, wherein the protein decomposition product comprises 20% by weight or more of a protein decomposition product having a weight average molecular weight of 1 to 2 kDa.

6. An immunotest kit comprising at least alkaline phosphatase or an alkaline phosphatase-labeled complex diluted with a stabilization solution comprising the stabilizer according to claim 1.

7. An immunotest kit comprising at least alkaline phosphatase or an alkaline phosphatase-labeled complex diluted with a stabilization solution comprising the stabilizer according to claim 2.

8. An immunotest kit comprising at least alkaline phosphatase or an alkaline phosphatase-labeled complex diluted with a stabilization solution comprising the stabilizer according to claim 3.

9. An immunotest kit comprising at least alkaline phosphatase or an alkaline phosphatase-labeled complex diluted with a stabilization solution comprising the stabilizer according to claim 5.

10. A method for preserving alkaline phosphatase or an alkaline phosphatase-labeled complex, the method comprising preserving alkaline phosphatase or the alkaline phosphatase-labeled complex in a stabilization solution comprising the stabilizer according to claim 1.

11. A method for preserving alkaline phosphatase or an alkaline phosphatase-labeled complex, the method comprising preserving alkaline phosphatase or the alkaline phosphatase-labeled complex in a stabilization solution comprising the stabilizer according to claim 2.

12. A method for preserving alkaline phosphatase or an alkaline phosphatase-labeled complex, the method comprising preserving alkaline phosphatase or the alkaline phosphatase-labeled complex in a stabilization solution comprising the stabilizer according to claim 3.

13. A method for preserving alkaline phosphatase or an alkaline phosphatase-labeled complex, the method comprising preserving alkaline phosphatase or the alkaline phosphatase-labeled complex in a stabilization solution comprising the stabilizer according to claim 4.

14. A method for preserving alkaline phosphatase or an alkaline phosphatase-labeled complex, the method comprising preserving alkaline phosphatase or the alkaline phosphatase-labeled complex in a stabilization solution comprising the stabilizer according to claim 5.

15. An immunotest method wherein an immunotest is performed using the immunotest kit according to claim 6.

16. An immunotest method wherein an immunotest is performed using the immunotest kit according to claim 7.

17. An immunotest method wherein an immunotest is performed using the immunotest kit according to claim 8.

18. An immunotest method wherein an immunotest is performed using the immunotest kit according to claim 9.
